(19) United States
(12) Patent Application Publication
(10) Pub. No.: US 2013/0261468 A1
(43) Pub. Date: Oct. 3, 2013

(54) Non-invasive portable dehydration diagnostic system, device and method

(71) Applicants: Herbert J. Semler, Portland, OR (US); Shawn Scott, Portland, OR (US)

(72) Inventors: Herbert J. Semler, Portland, OR (US); Shawn Scott, Portland, OR (US)

(73) Assignee: Semler Scientific, Inc.

(21) Appl. No.: 13/374,324

(22) Filed: Dec. 20, 2011

Related U.S. Application Data
(63) Continuation-in-part of application No. 12/317,538, filed on Dec. 24, 2008, now abandoned.
Application No. 12/317,538 is a continuation-in-part of application No. 09/971,507, filed on Oct. 4, 2001, now abandoned.
Application No. 09/971,507 is a continuation-in-part of application No. 12/001,505, filed on Dec. 11, 2007, now Pat. No. 7,628,760.
(60) Provisional application No. 61/459,898, filed on Dec. 20, 2010.

(51) Int. Cl. A61B 5/00; A61B 5/0205; A61B 5/026

(52) U.S. Cl. CPC A61B 5/4875; A61B 5/0261; A61B 5/7278; A61B 5/742; A61B 5/0004; A61B 5/0022; A61B 5/0205
USPC 600/473

(57) ABSTRACT

A non-invasive patient hydration monitoring system, device, and method are disclosed. The invented device utilizes a non-invasive photo-plethysmographic (PPG) finger- or toe-probe with an infrared transceiver to measure blood perfusion or circulation in an extremity. Such perfusion data is processed using correlation techniques into patient hydration data by a microprocessor and software application that preferably resides in a cell phone or similar portable hardware/firmware/software platform. Individual and successive patients can be quickly screened, baselined, diagnosed, and reported to identify individuals with dehydration conditions that are indicators of more important health issues such as disease and contagion.

### RELATED APPLICATIONS

This application is a continuation-in-part of and claims the benefit of priority from U.S. application Ser. No. 12/317,538 filed on Dec. 24, 2008 (which is a continuation-in-part of and claims the benefit of priority from U.S. application Ser. No. 09/971,507 filed on Oct. 4, 2001); this application further is a continuation-in-part of and claims the benefit of priority from U.S. application Ser. No. 12/001,505 filed on Dec. 11, 2007 (now U.S. Pat. No. 7,628,760); and this application further claims the benefit of priority from U.S. provisional application Ser. No. 61/459,898 entitled NON-INVASIVE PORTABLE DEHYDRATION DIAGNOSTIC SYSTEM, DEVICE AND METHOD FOR ITS USE, filed Dec. 20, 2010, the disclosures of which are all incorporated herein in their entirety by this reference.

### FIELD OF THE INVENTION

The invention relates generally to the field of detecting infectious disease conditions in patients. More particularly, the invention relates to non-invasively and objectively detecting patient dehydration using a portable diagnostic device.

### BACKGROUND OF THE INVENTION

Dehydration and low blood volume are closely correlated. A literature survey indicates that blood volume and dehydration are linked. Known references include: Partridge, Use of pulse oximetry as a noninvasive indicator of intravascular volume status, 3 J. OF CLINICAL MONITORING 264 (1987); Molochnyi, Changes in the peripheral circulation of children with severe forms of acute intestinal infections [translated], PEDIATRIIA (7-9): 20-4 (1992); Perel, et al., Systolic blood pressure variation is a sensitive indicator of hypovolemia in ventilated dogs subjected to graded hemorrhage, 67 ANESTHESIOLOGY 498 (1987); Pizov, et al., The use of systolic pressure variation in hemodynamic monitoring during deliberate hypotension in spine surgery, 2 J. OF CLINICAL ANESTHESIA 96 (1990); Shamir, et al., Pulse oximetry plethysmographic waveform during changes in blood volume, 82 BRITISH J. OF ANAESTHESIA 178 (1999), Burkert, et al., Non-invasive continuous monitoring of digital pulse waves during hemodialysis, 52 ASAIO J. 174 (2006); and Shelley, et al., WIPO patent application WO/2010/045556 entitled VOLUME STATUS MONITOR: PERIPHERAL VENOUS PRESSURE, HYPERVOLEMIA AND COHERENCE ANALYSIS. The last listed reference teaches invasive (standard intravenous or IV) techniques for analyzing ventilation-induced variation of waveforms in the peripheral vasculature.

A system and method for non-invasively measuring blood perfusion or circulation in an extremity are disclosed in U.S. Pat. No. 7,628,760 entitled CIRCULATION MONITOR AND MONITORING METHOD, which issued Dec. 8, 2009. That patent (subject to common ownership herewith by Semler Scientific, Inc. of Portland, Oreg.) teaches a non-invasive finger- or toe-probe sensor operatively coupled with an algorithmic computing element for producing an accurate measure of peripheral blood perfusion in a subject. The blood perfusion measurement may be represented as a normalized circulation index (CI), as described and illustrated therein. U.S. Pat. No. 7,628,760 is incorporated herein in its entirety by this reference.

### BRIEF DESCRIPTION OF THE DRAWINGS

FIG. 1 is a simplified schematic block diagram of the diagnostic device in accordance with one embodiment of the invention.

FIG. 2 is a schematic block and flow diagram of the software application that resides and executes within a dedicated-application microprocessor/memory portion of the cell phone or other portable computing platform shown in FIG. 1.

FIG. 3 is a flowchart illustrating the use of the invented device.

### DETAILED DESCRIPTION OF THE PREFERRED EMBODIMENTS

The invention involves a non-invasive, continuous Fluid Volume Monitor (“FVM”), implemented on a widely available, low-cost cell phone or other computing and telecommunications platform. The FVM would be used in both urban and rural areas of underdeveloped countries to determine patients' fluid status for the purpose of diagnosing dehydration resulting from malnutrition and disease rampant in these areas, e.g. cholera, dysentery, etc. The FVM's object is to provide a low-cost, simple tool for diagnosing dehydration and preventing hypovolemic shock, and for providing modified fluid recommendations based on the FVM's data.

There is an urgent need for an easy, non-invasive and objective test for identifying dehydration in its early stages, since current methods depend on symptoms and physical findings such as cold hands, weak and fast pulse, dizziness, lethargy, and thirst, which are all subjective. Verification of dehydration requires blood tests such as hemoglobin and hematocrit to indicate hemo-concentration which may not be specific in diagnosing dehydration. The FVM, by virtue of its implementation on off-the-shelf cellular platforms and its non-invasive use model, enables a portable, low-cost, easily deployable device for assisting health or other authorities such as clinicians more accurately to screen for and to diagnose dehydration. This unique multi-functional device will enable clinicians to make speedier and more accurate diagnoses and to communicate these diagnoses to health or other authorities, thereby hastening time to therapy and reducing contagious outbreaks of underlying disease.

The FVM is based on a patented, non-invasive photo-plethysmographic (PPG) device incorporating an electro-optical sensor and a digital algorithm. This FDA-cleared device has very recently been introduced for clinical use in measuring a patient's peripheral blood flow/volume for the purpose of monitoring various conditions including peripheral arterial disease. The device includes a pulsatile blood flow/volume detector that uses infrared (IR) light to detect blood flow changes in a patient's extremity (e.g. a digital extremity such as a finger and/or toe or another suitable extremity such as a penis). It obtains optical density measurements by intermittently providing a current pulse of known amplitude to the IR emitter, which sends IR light through a patient's body tissue, typically a finger or toe. The transmitted light, attenuated by variations in blood flow and blood components in the body tissue, is measured and digitized. A proprietary signal processing algorithm using power spectral density distributions calculates values from the data that represent the blood volume changes. This information is used to make diagnoses regarding disease states such as peripheral artery disease. There are no other similar devices currently available. Configurations using a sensor attached to a WINDOWS®-based computer are currently being marketed to physicians, and versions may be adapted to other platforms, e.g. WINDOWS® mobile computing, ANDROID™, etc.

The FVM is expected to operate similarly to the current blood circulation monitoring diagnostic device, and would comprise a sensor operatively connected (e.g. physically via a cable or wirelessly via a BlueTooth protocol) to a cell phone or other mobile computing and telecommunications platform. The operator would place the sensor onto the patient's fingertip, where it would collect optical data about blood flow/volume. The algorithm, embedded in cell phone read-only memory (ROM), would calculate the dehydration status and display this information to the operator, which could then also be transmitted back to a remote health authority. Because the sensor draws little current, it enables maximum battery life for use in non-hospital urban and rural, e.g. field, locations.

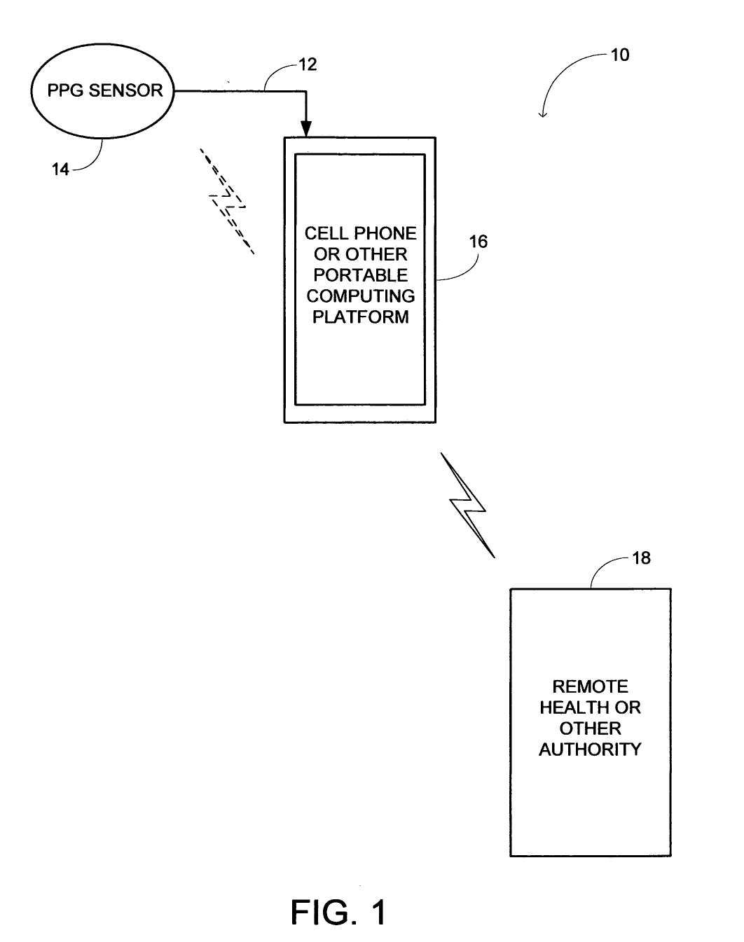

The configuration of the invented FVM device 10 includes two slightly different embodiments illustrated in FIG. 1: A first version includes a wire cable 12 connecting a PPG sensor 14 (which is placed on a patient's finger, toe, or other extremity) to a cell phone or other portable computing and telecommunications platform 16. Wire cable 12 may take the form of a USB cable connected between sensor 14 and a USB or other hard-wired telecommunications port on cell phone 16. In contemplation of easier and quicker deployment, a second version replaces wire cable 12 connecting PPG sensor 14 to cell phone 16 with a Bluetooth or other suitable wireless connection (illustrated in dashed lines in FIG. 1). Cell phone 16 is linked in accordance with one embodiment of the invention to a remote health or other authority 18, as indicated. Those of skill in the art will appreciate that such can be via satellite and/or cell phone tower and/or other suitable wireless conveyance (not shown in FIG. 1 for the sake of clarity).

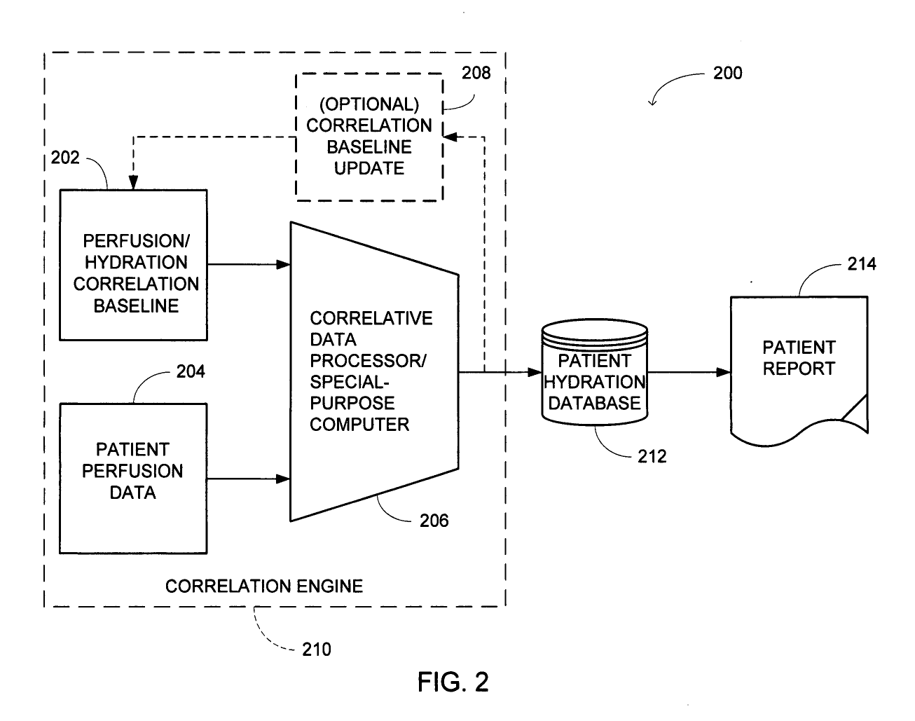

(Those of skill in the art will appreciate from the discussion herein that a dedicated and proprietary Internet server, a cloud server, or a flash memory device may also be operatively connected to cell phone 16. Such would provide what is described below by reference to FIG. 2 as a patient hydration database for archiving of patient hydration data at a secure location that is remote from cell phone 16. But those of skill also will appreciate that such a database alternatively can be maintained in a SIM card, memory stick, or other non-volatile memory device within or intimately connected with cell phone 16. Indeed, those of skill in the art will appreciate that cell phone 16 may, within the spirit and scope of the invention, be augmented by an external memory storage device or other peripheral circuitry serving any auxiliary function that usefully extends the functionality, accuracy, or security of the patient hydration data retrieval, monitoring, processing, and/or storage. Nevertheless, in accordance with one embodiment of the invention, it is believed that a modestly memory-equipped cell phone or other portable computing platform 16 alone suffices to provide useful patient hydration data gathering, processing, and storage capability.)

FIG. 2 is a schematic block and flow diagram of the application software mechanism 200 residing on and executing within a processor and instruction-store (memory) of portable computing platform 16 shown in FIG. 1. Software 200 includes a blood perfusion/body hydration correlation baseline data store 202 that is typically empirically derived for a given patient population. Those of skill in the art will appreciate that such correlation baseline data store 202 may be represented by data taking any desired form, e.g. a look-up table, a numeric linear or non-linear function F(n), or any other data and/or arithmetic form suitable for storage in a memory device (not shown) in computing platform 16 and suitable for processing in accordance with suitable processing parameters.

Software mechanism 200 further includes patient perfusion data store 204 obtained from PPG sensor 14 of device 10. Those of skill also will appreciate that patient perfusion data store 204 can also take any suitable form, and in accordance with one embodiment of the invention is stored in a memory (not shown but typically residing within the guts of portable computing platform 16). Patient perfusion data store 204 will be understood by those of skill in the art to be patient-specific, whereas perfusion/hydration correlation baseline data store 212 will be understood to be either patient-specific or patient-normal, e.g. representing an entire population or population group representing an ethnic, cultural, geographic, seasonal, or other prevailing norm.

Software mechanism 200 further includes a correlative data processor or special-purpose computer 206 configured typically with software instructions stored in memory for execution by a microprocessor to input the baseline data and the patient data and to produce a hydration index therefrom. Those of skill in the art will appreciate that correlative data processor 206 can be programmed in any suitable way accurately and repeatably to represent a specific patient's hydration by derivation and/or calculation (e.g. interpolation, extrapolation, etc.) from the patient's measured perfusion. Any suitable software architecture, programming language, data structures, algorithms, and coding particulars can be used, as are known.

Optionally, patient hydration data output from correlative date processor 206 can be used to update perfusion/hydration correlation baseline data store 202 more accurately to reflect a particular patient's determined perfusion/hydration correlation. Such is illustrated in FIG. 2 by (optional) correlation baseline update block 208, which takes output from correlative data processor 206, processes the data therefrom, and produces one or more adjustment inputs to perfusion/hydration correlation baseline 202. Those of skill in the art will appreciate that update block 208 can be implemented in any suitable way such as modifying baseline initial conditions, starting values, look-up table values, or other constants, variables, or arithmetic formulae, thereby to render the baseline data store more accurately reflective of a specific patient's perfusion/correlation baseline, which will be understood to be the theoretical model by which perfusion data is interpreted as hydration data. Nevertheless, applicants do not intend to be held to any particular theory of operation, and instead submit their claims are limited only by their own structural and functional language as broadly contemplating any operational theory.

Perfusion/hydration correlation baseline data store 202, patient perfusion data store 204, correlative data processor/special-purpose computer 206 and (optional) correlation baseline update block 208 will be referred to collectively herein as a patient hydration-derivation engine 210. Alternatively, hydration-derivation engine 210 may be referred to herein as correlation engine 210.

Those of skill in the art will appreciate that hydration-derivation or correlation engine 210 may be implemented in any suitable alternative form to that illustrated in FIG. 2. For example, functions and functional blocks shown herein may be added, deleted, combined, re-ordered, separated or otherwise partitioned based upon design choice. Those of skill in the art also appreciate that functional blocks may have different functional attributes or descriptors or characteristics or configurations, may operate on different inputs and generate different outputs, and may be operatively coupled in alternative ways. Such alternative implementations are contemplated as being within the spirit and scope of the invention, which scope is limited only by the appended claims.

FIG. 2 illustrates that the output of correlative data processor/special-purpose computer 206 also is input to a patient hydration database 212, which in accordance with one embodiment of the invention stores one or more (typically serial) patient-specific hydration data records for use in reporting, archiving, trend-analyzing, etc. Those of skill in the art will appreciate finally that a patient report 214 in accordance with one embodiment of the invention is output from patient hydration database 212 in any suitable form. For example, the report may be in hard-copy or intangible form, and/or may be permanent or transient, and/or may be delivered to a local or remote site, and/or may be in raw data, textual, and/or graph form. The patient hydration data itself may be represented in any suitably useful units of measure from percentage of norm to water weight, to body fluid index, to fluid mass, to a more interpretive body hydration index that represents the patient's hydration in an objective scale, for example, from 1 (dangerously low) to 6-7 (low-normal and thus cautionary) to 8-10 (safely normal).

This index representation of the patient's hydration data may be referred to herein as the patient's hydration index (HI). Those of skill will appreciate that a patient's HI is correlated with the patient's CI described in the above-referenced CIRCULATION MONITORING SYSTEM patent as the patient's circulation index. Indeed, in accordance with one embodiment of the present invention, a patient's HI is derived from the patient's CI.

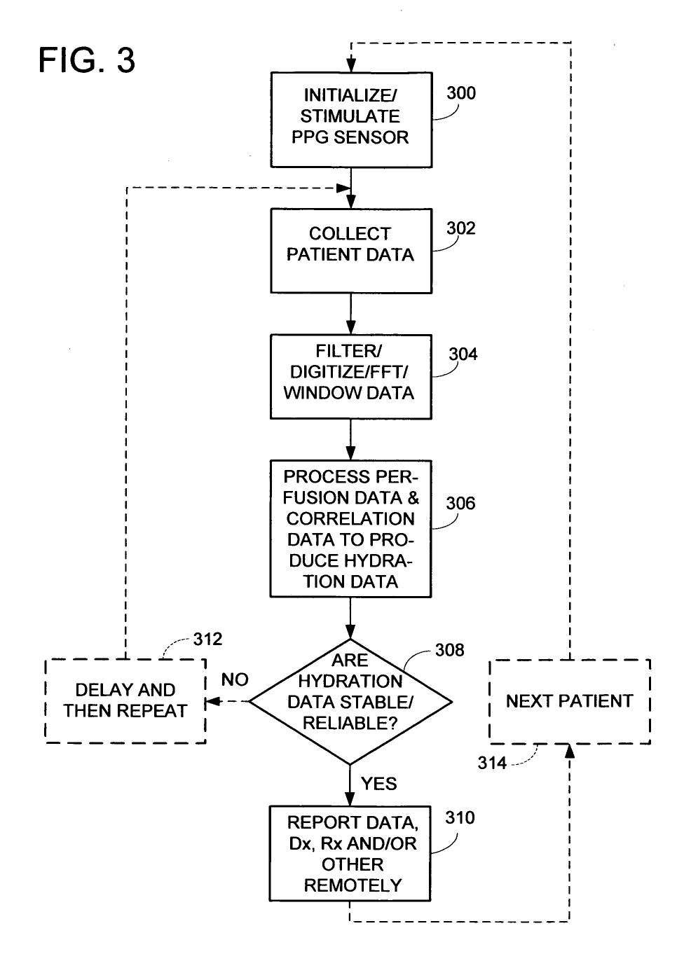

FIG. 3 is a flowchart illustrating the use of the invented device. Use of the invented device begins at block 300, wherein PPG sensor 16 is initialized and stimulated in accordance with the teachings of the CIRCULATION MONITORING SYSTEM patent referenced hereinabove. At block 302, patient perfusion (or circulation) data are collected also in accordance with the patented teachings. At block 304, the collected patient perfusion data are filtered, digitized, transformed as by use of a Fast Fourier Transform (FFT), and windowed (as by use of a Hamming or preferably Blackman window, for example), also in accordance with the patented teachings. At block 306, the collected, filtered, digitized, transformed, and windowed data are further processed with stored correlation data, as described above with respect to correlation engine 210. At block 308, it is determined based upon predefined (and typically stored) stability/reliability criteria whether the processed data are sufficiently stable and thus reliable to report. If so, then at block 310, raw or processed data, diagnostic (Dx) and/or prescriptive (Rx) patient data, or other data are reported, for example, to a remote observer such as a clinician or a health authority.

If it is determined at block 308 that the processed data are unstable and thus potentially unreliable, then at block 312, a suitable delay (which may be a zero time delay but which typically is more, e.g. 15 seconds or 1 minute or more) is imposed and the patient data collection and subsequent process steps are repeated until such time as the data are deemed sufficiently stable and thus reliable to report. Those of skill in the art will appreciate that accurate hydration monitoring typically is a serial process rather than a point-of-time or so-called snapshot in time action. This is because, like perfusion (or circulation), hydration is more of a dynamic than static condition. Thus, those of skill in the art will understand appropriate time delays and the collection of serial data records to establish baseline measurements that accurately represent a patient's present and continuing or perhaps improving or deteriorating hydration condition.

As described above, a patient's hydration can be reported in any appropriate form and more or less artificial intelligence (AI) can go into the reporting. Thus, the software application that operates on portable hardware platform 16 can by suitable artificial intelligence techniques render simple or complex reports that might involve simple reporting of a patient's hydration index (HI) or might more precisely quantify a hydration (or dehydration) condition of the patient, provide graphs of measured trend lines (important for triage and diagnosis) and even might predict further trends if the dehydration condition remains untreated. The software application also might provide more than diagnostic reports but might also prescribe appropriate remedial actions or treatments, subject to second opinions from attendant personnel or remote clinicians or health authorities.

Those of skill in the art will appreciate that the software architecture described and illustrated herein can be implemented in any suitable code by the use of any suitable coding and language tools. For example, any one or more of C++, XML, Flash, Actionscript, and SQL are a suitable suite of tools for coding the invented system and device software.

After a given patient's hydration condition is reported, a next patient may be processed via block 314 by repeating all process steps with newly acquired data to provide a succession of processed patients and reports enabling attendant personnel to quickly and accurately screen, diagnose, and treat patients exhibiting symptoms of dehydration. Those of skill in the art will appreciate that the invented device facilitates such screening, diagnosis, and even treatment by its ubiquity, portability, ease of use, simplicity, wirelessness, and accuracy. Moreover, the use of the invented device is non-invasive, thus avoiding common objections to invasive techniques such as syringes, IVs, and other subcutaneous patient privacy and security invasions. Utility is in the use of smart phones, which are probably the most widely distributed computing platform in the world and are inherently networked. Smart phones will be understood by those of skill in the art to be particularly suited for regions without land-line infrastructure. Moreover, the non-invasive approach described, illustrated, and claimed herein is also safer considering the risk of access-site infection that characterizes conventional invasive monitoring means. In addition, the use of a specialized sensor, algorithm, and database permit more precise diagnosis with little or no medical training for attendants, clinicians, et al.

Those of skill in the art will appreciate that low blood perfusion correlates with low hydration in a monitored patient. Thus, it is believed that the invented use of the non-invasive portable device described above to monitor and diagnose patient dehydration has great utility in the early detection of potentially life-threatening infectious disease in patients exhibiting dehydration, and in avoidance of potentially life-threatening contagion of others.

Those of skill in the art will appreciate that the so-called report may be tele-communicated remotely to an archival store server on the wide-area network (WAN) such as the world-wide web, to a particular health care database server, to a hospital or doctor of record, to a desired clinic, or to a personal computer or one connected to a local area network (LAN). Such tele-communciation can take any suitable form and use any suitable technique and/or equipment including data packetization, satellite, cell tower, repeater station, and/or proprietary or open web server, e.g. a cloud server.

Patient data security measures are contemplated as being also within the spirit and scope of the invention to meet regulatory requirements such as HPPA regulation in the United States. Current or future data encryption standards may be used, as are known, as may be patient and/or user names or identification (ID) codes such as Social Security numbers (SSNs), passwords, and/or personal identification numbers (PINs), also as are known. Such patient data security measures are especially important in the global patient disease and/or condition monitoring application contemplated by the present invention.

Those of skill in the art will appreciate that the invented device also permits patients equipped with a Smartphone (e.g. an iPHONE®, an ANDROID®, a BLACKBERRY®, etc.) to self-monitor and diagnose. Thus in accordance with one embodiment of the invention, a display of cell phone 16 becomes a windowed user interface, as is known in the world of software applications or so-called “apps”, into the patient's own state of hydration. The display may be used to provide patient report 14 in a “soft” form for viewing and studying by the patient or an attendant. Such a display may provide user input controls such as buttons for starting and stopping and monitoring of the status of the PPG sensor and associated data processing software, as well as user outputs such as status indicators, tabulated data, HI results, waveforms representing the same, trend-line graphs, selected group norm comparisons, percentiles, and other interpretive and perhaps extremely helpful outputs of any suitable form.

Notwithstanding the above, the invented device finds particular utility in third-world countries and field deployment for masses of people who may suffer malnutrition or disease as a result of drought, flooding, infestation, and exposure to environmental contaminants. Thus, the device's lightweight portability, ease of use, repeatability, and accuracy enable a new approach to global disease identification and eradication while keeping the populations of the world in better overall health. It also enables health authorities to early detect and avoid endemic disease and to develop infrastructure for better handling and, in the future, avoiding provincial or global health crises.

It will be understood that the present invention is not limited to the method or detail of construction, fabrication, material, application or use described and illustrated herein. Indeed, any suitable variation of fabrication, use, or application is contemplated as an alternative embodiment, and thus is within the spirit and scope, of the invention.

It is further intended that any other embodiments of the present invention that result from any changes in application or method of use or operation, configuration, method of manufacture, shape, size, or material, which are not specified within the detailed written description or illustrations contained herein yet would be understood by one skilled in the art, are within the scope of the present invention.

Finally, those of skill in the art will appreciate that the invented method, system and apparatus described and illustrated herein may be implemented in software, firmware or hardware, or any suitable combination thereof. Preferably, the method system and apparatus are implemented in a combination of the three, for purposes of low cost and flexibility. Thus, those of skill in the art will appreciate that embodiments of the methods and system of the invention may be implemented by a computer or microprocessor process in which instructions are executed, the instructions being stored for execution on a computer-readable medium and being executed by any suitable instruction processor.

Accordingly, while the present invention has been shown and described with reference to the foregoing embodiments of the invented apparatus, it will be apparent to those skilled in the art that other changes in form and detail may be made therein without departing from the spirit and scope of the invention as defined in the appended claims.

We claim:

1. A non-invasive human-patient fluid volume monitor comprising:
a non-invasive electro-optical detector configured to monitor a human patient's pulsatile blood flow, the detector including an infrared (IR) light to detect blood flow changes in an extremity of the patient;
a digital computing element including a digital processor and a memory for storing and instructions and data, the digital computing element being configured to be operatively coupled with the detector to monitor the patient's pulsatile blood flow and to calculate a fluid volume measurement therefrom; and
a display coupled with the digital computing element, the display configured to present human-patient fluid volume data thereon in human-readable report format.

2. The monitor of claim 1, wherein the digital computing element includes a first algorithm structure configured to produce a data stream representing the patient's pulsatile blood flow in the form of a circulation index (CI), and wherein the digital computing element further includes a second algorithm structure configured to produce from the CI a data stream representing the patient's fluid volume in the form of a hydration index (HI).

3. The monitor of claim 2, wherein the electro-optical detector is coupled to the digital computing element via one or more wired signal conveyances.

4. The monitor of claim 2, wherein the electro-optical detector is coupled to the digital computing element via a wireless conveyance.

5. The monitor of claim 2, wherein the digital computing element and the memory are contained within a portable computer platform that includes one or more of a personal computer (PC), a laptop computer, a notebook computer, a personal digital assistant (PDA), and a cell phone.

6. The monitor of claim 5 further comprising:
a telecommunications mechanism operatively coupled with the digital computing element, the telecommunications mechanism being configured to transmit dehydration status information to a remote health authority.

7. The monitor of claim 2, wherein the memory includes a blood perfusion/body hydration correlation baseline data store and a patient perfusion data store, and wherein the digital computing element is configured to derive a patient's HI from the patient's CI via one or more of a look-up table, a numeric linear or non-linear function (F), and any other suitable data and arithmetic form suitable for storage in the memory.

8. A non-invasive human-patient dehydration diagnostic system comprising:
a non-invasive electro-optical detector configured to monitor a human patient's pulsatile blood flow, the detector including an infrared (IR) light to detect blood flow changes in an extremity of the patient;
a patient hydration-derivation engine operatively coupled with the detector, the engine including a patient perfusion data store for storing pulsatile blood flow data from the detector and a correlative data processor for deriving patient hydration data from patient perfusion data and for storing the same in a memory; and
a report mechanism operatively coupled with the engine for reporting one or more of the patient's perfusion data and the patient's derived hydration data in a human-readable report format.

9. The system of claim 8, wherein the engine includes one or more look-up tables or one or more mathematical formulae configured to produce the derived patient hydration data from the monitored patient perfusion data.

10. The system of claim 8, wherein at least the engine and the report mechanism are contained within a portable computer platform that includes one or more of a personal computer (PC), a laptop computer, a notebook computer, a personal digital assistant (PDA), and a cell phone.

11. The system of claim 10 further comprising:
a telecommunications mechanism operatively coupled with the digital computing element, the telecommunications mechanism being configured to transmit dehydration status information to a remote health authority.

12. The system of claim 8, wherein the detector and the engine are operatively coupled via one or more wired signal conveyances.

13. The system of claim 8, wherein the detector and the engine are operatively coupled via a wireless conveyance.

14. The system of claim 8, wherein the engine further includes a patient perfusion/hydration correlation baseline data store configured to store patient-specific correlation data, and wherein the engine further includes a correlation baseline data update mechanism configured to input patient perfusion/hydration correlation data from the correlative data processor, to process the correlation data therefrom, and to produce one or more adjustment inputs to the patient perfusion/hydration correlation baseline data store.

15. The system of claim 14, wherein the engine is configured to determine whether the processed data meet defined stability/reliability criteria and, if not, then to impose a defined delay and thereafter to repeat patient data collection and processing steps until such criteria are met.

16. A non-invasive human-patient dehydration diagnostic method comprising:
placing a photo-plethysmographic (PPG) sensor on a patient's extremity;
monitoring the patient's pulsatile blood blow through the extremity to produce a patient blood flow data stream;
deriving a patient hydration data stream from the blood flow data stream, the deriving being performed by a correlation engine;
storing the patient hydration data stream in a memory; and
reporting the stored patient hydration data stream in human-readable form.

17. The method of claim 16, wherein the monitoring, deriving and reporting steps are performed electronically using one or more of analog and digital signal processing and storing of data produced by the processing.

18. The method of claim 17 which further comprises:
determining whether the derived patient hydration data stream meets defined stability/reliability criteria and, if not, then before the reporting step imposing a defined delay and thereafter repeating the monitoring, deriving, and storing steps until such criteria are met.

19. The method of claim 17, wherein the reporting step includes displaying the patient hydration data stream in one or more of a raw data, textual, and graph form.

20. The method of claim 19, wherein at least the deriving, storing, and displaying steps are performed by application software instructions residing in a memory and executing in a digital processor embedded within a cell phone.
